(19) United States
(12) Patent Application Publication
(10) Pub. No.: US 2012/0310048 A1
(43) Pub. Date: Dec. 6, 2012

(54) EXPANDING CONDUITS

(75) Inventors: Tzony Siegal, Shoeva (IL); Oded Loebl, Tel Mond (IL); Didier Toubia, Raanana (IL)

(73) Assignee: NLT-SPINE LTD, KFAR SABA (IL)

(21) Appl. No.: 13/578,818

(22) PCT Filed: Feb. 15, 2011
(86) PCT No.: PCT/IB11/50622

§ 371 Date: Aug. 14, 2012

Related U.S. Application Data
(60) Provisional application No. 61/304,506, filed on Feb. 15, 2010.

(51) Int. Cl. A61B 1/32

(52) U.S. Cl. 600/206

(57) ABSTRACT

A device comprising an elongated conduit comprising further at least an upper profile and at least a lower profile is disclosed. The upper and lower profiles being relatively displaceable such that when a distal part of the conduit is deployed between two regions of tissue, a spacing between an upper distal surface of the upper profile and a lower distal surface of the lower profile can be varied, thereby opening a space between the two regions of tissue, and wherein the upper and lower profiles are further configured so as to define therebetween a working channel to provide access to the space between the two regions of tissue through the conduit.

### FIELD OF THE INVENTION

The invention relates generally to surgical tools and conduits, and more particularly to expanding conduits and methods for creating a surgical working channel using such expanding conduits.

### BACKGROUND OF THE INVENTION

A wide range of surgical procedures require a step of distracting tissue in which two types of tissue, or two regions within the same tissue, are pushed apart to open a space to facilitate performance of subsequent steps of the procedure. In a non-limiting example of spinal surgery, it is often desired to increase a spacing between adjacent vertebral bodies for the purpose of accessing the intervertebral space during procedures performed to the intervertebral disc, total disc replacements or vertebral fusion procedures. In many cases, the distraction “step” is actually a non-trivial process, requiring a series of actions with distractor tools of different sizes.

Subsequent to the distraction step, it is often desirable to provide a conduit to define a working channel for introduction of tools and/or implants and/or biocompatible materials, or for removal of tissue or other material from the body.

Various non-limiting examples of devices for introduction into a body in a straight configuration via a conduit and assuming within the body a predefined curved configuration have been disclosed by the applicant in PCT patent application publication no. WO2006/072941. The device included an elongated element formed from a number of segments interconnected so as to form effective hinges therebetween. The elongated element is introduced in a straight configuration within a conduit and assumes a curved configuration when deployed. A wide selection of applications of such devices, particularly in the field of spinal surgery, are described therein.

It would be highly advantageous if the steps of distraction and providing a conduit could be performed simultaneously by a single device, thereby rendering easier and more efficient a wide range of surgical procedures. Specifically, it would be advantageous to provide expanding conduits to create space between two tissue regions and to provide an access channel to the created space between the two regions of tissue through the elongated conduit.

### SUMMARY OF THE INVENTION

According to an embodiment of the present invention, there is provided an elongated conduit including at least an upper profile and at least a lower profile. The upper and lower profiles being relatively displaceable such that when a distal part of the conduit is deployed between two regions of tissue, a spacing between an upper distal surface of the upper profile and a lower distal surface of the lower profile can be varied, thereby opening a space between the two regions of tissue, and wherein the upper and lower profiles are further configured so as to define therebetween a working channel to provide access to the space between the two regions of tissue through the conduit.

According to a further feature of an embodiment of the present invention, an expansion mechanism is removably deployable within the conduit and configured to apply outward forces between the upper and lower profiles so as to expand the elongated conduit, thereby increasing a spacing between the two regions of tissue is provided. The expansion mechanism is deployable within the working channel to apply outward forces between the upper and lower profiles and is subsequently removed to make the working channel available to access the space between the two regions of tissue.

According to a further feature of an embodiment of the present invention, a locking arrangement is configured to lock the upper and lower profiles to maintain an expanded configuration of the elongated conduit after the expansion mechanism is removed is provided. The locking arrangement is configured to be releasable by relative motion of the upper and lower profiles parallel to a length of the elongated conduit, thereby allowing contraction of the elongated conduit for ease of removal.

According to a further feature of an embodiment of the present invention, the elongated conduit further comprises one or more additional profiles deployed to span a gap between the upper profile and the lower profile in an expanded configuration of the elongated conduit.

According to a further feature of an embodiment of the present invention, the expansion mechanism may be an inverted scissor like mechanism having a rotation axis in a mid portion of the elongated conduit, and wherein applying a force onto one side of the conduit separate the tips on the other side of the elongated conduit enlarging the working channel.

The expansion mechanism may be a cam wedge mechanism comprised of at least one component with a curved surface, an upper and lower rods and connecting pins, and wherein pulling in or pushing out one of the rods creates a rotation of the curved component around the pins and wherein the rotation of the component increases the height of the curved component in the direction perpendicular to the axis of motion and applies force outwards onto the upper and the lower elongated conduit profiles enlarging the working channel.

The expansion mechanism may be a jack mechanism comprised of at least one set of a handle attached to a threaded rod, a block fixed on the rod, a block that moves relative to the rod, a block that contacts the upper conduit profile and two connector shafts between the blocks, and wherein turning the handle advances the movable block toward the fixed block causing the central block to move in the perpendicular direction to the blocks pushing outwards the upper and lower profiles and enlarging the working channel.

The expansion mechanism may be a spring mechanism comprised of a threaded rod connected to a handle, an elastic material curved on the threaded rod having one or more arches that come in contact with the upper and lower profiles of the conduit and where one end of the curved elastic material is connected to the end of the rod such that the rod has a rotational axis of freedom and the other end of the curved elastic material is connected to a threaded block, and wherein turning the block compresses the curved elastic material pushing outwards the upper and the lower conduit profiles enlarging the working channel.

According to a further feature of an embodiment of the present invention, the upper and lower profiles are hingedly engaged such that pressing together of proximal ends of the upper and lower profiles results in moving apart of distal ends of the upper and lower profiles.

According to a further feature of an embodiment of the present invention, the elongated conduit has a substantially rectangular cross-section.

According to a further feature of an embodiment of the present invention, the elongated conduit has a locking arrangement configured to lock the upper and lower profiles in an expanded configuration.

According to an embodiment of the present invention, there is provided a method for accessing a volume within a body comprising the steps of (a) inserting into the body an elongated conduit comprising at least an upper profile and a lower profile, (b) applying outward forces between the upper and lower profiles so as to expand the elongated conduit, thereby increasing a spacing between two regions of tissue and opening a space therebetween, and (c) accessing the space between the two regions of tissue through the elongated conduit is provided.

According to a further feature of an embodiment of the present invention, the step of applying outward forces is performed using an expansion mechanism removably deployable within the elongated conduit.

According to a further feature of an embodiment of the present invention, the expansion mechanism is removed subsequent to applying outward forces so as to make available a working channel through the elongated conduit to access the space.

According to a further feature of an embodiment of the present invention, tissue is removed through the elongated conduit wherein the removed tissue may be tissue of an intervertebral disc.

According to a further feature of an embodiment of the present invention, implants are introduced into the space through the elongated conduit.

According to a further feature of an embodiment of the present invention, biocompatible material is introduced into the space through the elongated conduit.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1a illustrates an expanding conduit, according to embodiments of the present invention;

FIG. 1b illustrates an expanding conduit in its open configuration, according to embodiments of the present invention;

FIG. 2a illustrates a telescopic expanding conduit in a closed configuration, according to embodiments of the present invention;

FIG. 2b illustrates a telescopic expanding conduit in a partially open configuration, according to embodiments of the present invention;

FIG. 2c illustrates a telescopic expanding conduit in a fully open configuration, according to embodiments of the present invention;

FIG. 3 illustrates an inverted scissor like expanding conduit in a closed configuration, according to embodiments of the present invention;

FIG. 4 illustrates a side view of the inverted scissor like expanding conduit in a closed configuration, according to embodiments of the present invention;

FIG. 5 illustrates the tip shape of the inverted scissor like expanding conduit in a closed configuration, according to embodiments of the present invention;

FIG. 6 illustrates another tip shape of the inverted scissor like expanding conduit in a closed configuration, according to embodiments of the present invention;

FIG. 7 illustrates an inverted scissor like actuation mechanism of an expanding conduit in an open configuration, according to embodiments of the present invention;

FIG. 8 illustrates a side view of the inverted scissor like actuation mechanism of an expanding conduit in an open configuration, according to embodiments of the present invention;

FIG. 9 illustrates a cam wedge actuation mechanism of an expanding conduit design in a closed configuration, according to embodiments of the present invention;

FIG. 10 illustrates the cam wedge actuation mechanism of an expanding conduit design in an open configuration, according to embodiments of the present invention;

FIG. 11 illustrates a multiple cam wedge actuation mechanism of an expanding conduit in a closed configuration, according to embodiments of the present invention;

FIG. 12 illustrates the multiple cam wedge actuation mechanism of an expanding conduit in an open configuration, according to embodiments of the present invention;

FIG. 13a illustrates a jack actuation mechanism of an expanding conduit in a closed configuration, according to embodiments of the present invention;

FIG. 13b illustrates a multiple jack actuation mechanism of an expanding conduit in a closed configuration, according to embodiments of the present invention

FIG. 14a illustrates the jack actuation mechanism of an expanding conduit in an open configuration, according to embodiments of the present invention;

FIG. 14b illustrates the multiple jack actuation mechanism of an expanding conduit in an open configuration, according to embodiments of the present invention;

FIG. 15 illustrates a cross section of the jack actuation mechanism inside an expanding conduit in a closed configuration, according to embodiments of the present invention;

FIG. 16 illustrates a cross section of the jack actuation mechanism inside an expanding conduit in an open configuration, according to embodiments of the present invention;

FIG. 17 illustrates a cross section of the cam wedge actuation mechanism inside an expanding conduit in a closed configuration, according to embodiments of the present invention;

FIG. 18 illustrates a cross section of the cam wedge actuation mechanism inside an expanding conduit in an open configuration, according to embodiments of the present invention;

FIG. 19 illustrates a spring actuation mechanism of an expanding conduit in an open configuration, according to embodiments of the present invention;

FIG. 20 illustrates the spring actuation mechanism of an expanding conduit in a closed configuration, according to embodiments of the present invention;

FIG. 21 illustrates a cross section of the spring actuation mechanism inside an expanding conduit in a closed configuration, according to embodiments of the present invention;

FIG. 22 illustrates the cross section of the spring actuation mechanism inside an expanding conduit in an open configuration, according to embodiments of the present invention.

FIGS. 23a-c illustrate a locking arrangement between the upper and the lower profiles of an expanding conduit, according to embodiments of the present invention.

FIGS. 24a-c illustrate another locking arrangement between the upper and the lower profiles of an expanding conduit, according to embodiments of the present invention.

FIGS. 25a-e illustrate a locking arrangement of an inverted scissor like expanding conduit, according to embodiments of the present invention.

FIGS. 26a-b illustrates another locking arrangement between the upper and the lower profiles of an expanding conduit, according to embodiments of the present invention.

FIGS. 27a-c illustrate a locking arrangement with one additional profile deployed to span a gap between the upper profile and the lower profile, according to embodiments of the present invention.

FIGS. 28a-d illustrate an external scissor expansion mechanism and an expanding conduit, according to embodiments of the present invention.

### DETAILED DESCRIPTION OF THE PREFERRED EMBODIMENTS

Certain embodiments of the present invention provide an expanding conduit including at least an upper profile and a lower profile, the upper and lower profiles being relatively displaceable such that, when a distal part of the conduit is deployed between two regions of tissue, a spacing between an upper distal surface of the upper profile and a lower distal surface of the lower profile can be varied, thereby opening a space between the two regions of tissue. The upper and lower profiles are further configured so as to define therebetween a working channel to provide access to the space between the two separated regions of tissue through the elongated conduit.

In certain embodiments, an expansion mechanism is removably deployable within the conduit and configured to apply outward forces between the upper and lower profiles so as to expand the elongated conduit, thereby increasing the spacing between the two regions of tissue. The expansion mechanism is deployable within the working channel to apply outward forces between the upper and lower profiles and is preferably subsequently removed to make the working channel available to access the space between the two regions of tissue.

The elongated conduit upper and lower profiles may have inter locking arrangement that prevents the expanding conduit from folding back after the expansion mechanism is removed. Furthermore, the elongated conduit may have one or more additional profiles in order to enlarge the elongated conduit's working channel.

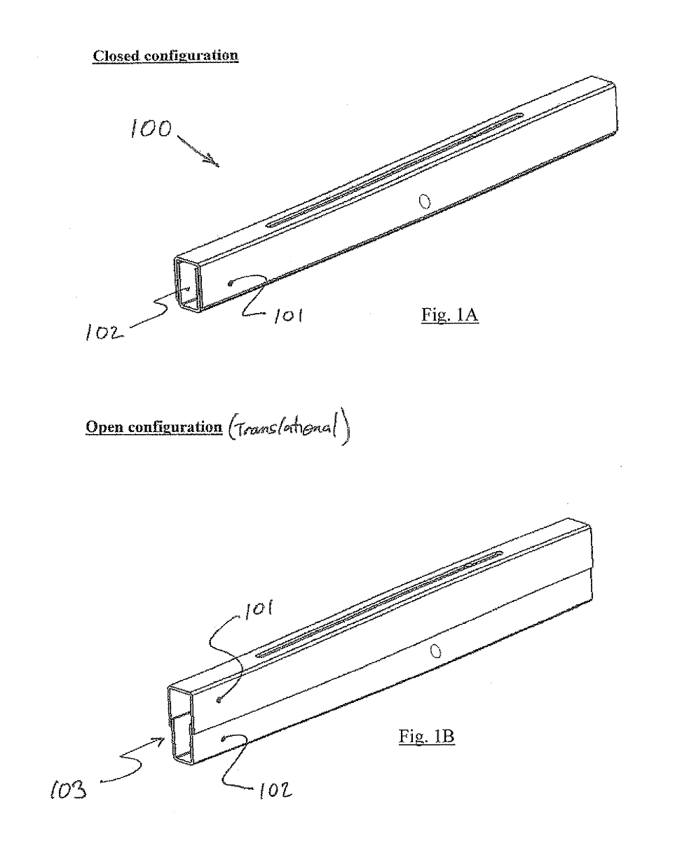

FIG. 1a illustrates an expanding conduit, according to embodiments of the present invention. The expanding conduit 100 is formed at least in part by upper 101 and lower 102 profiles, typically with a U-shaped cross-section, that inter-engage, typically with one fitting into the other (shown also in FIG. 5 and FIG. 6), leaving an open working channel preferably roughly along the center.

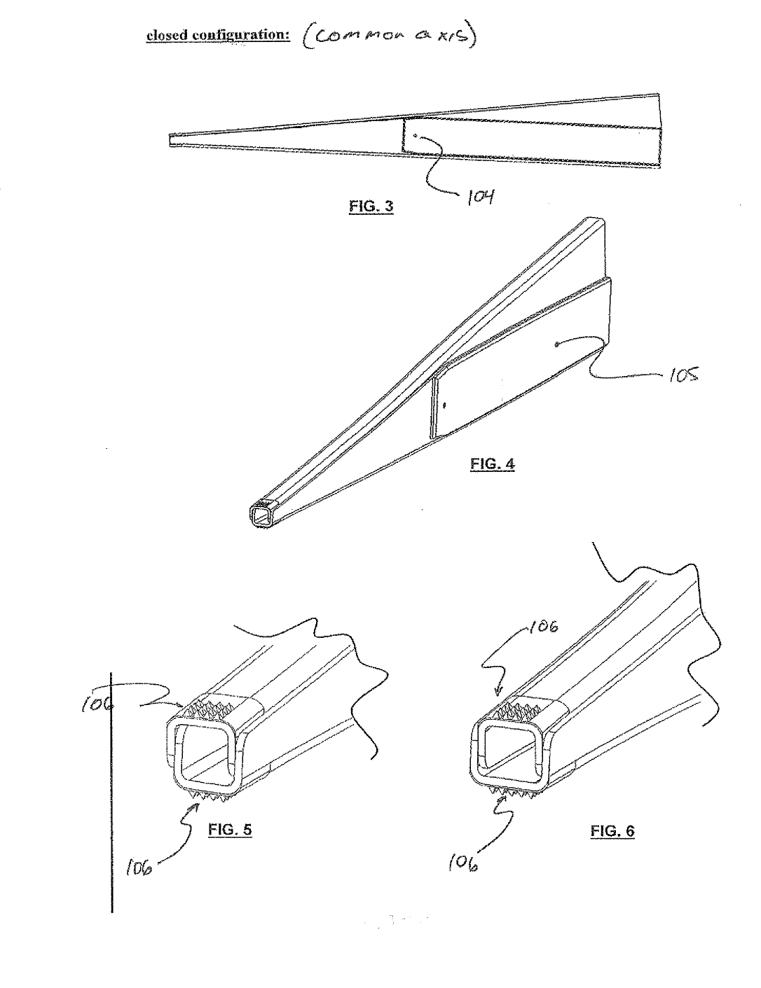

FIG. 1b illustrates an expanding conduit in its open configuration, according to embodiments of the present invention. The upper profile 101 and lower profile 102 may move relative to each other thus enlarging the working channel 103. Surgery tools and implants may be deployed through working channel 103. The motion between the profiles can be translational (moving up or down in parallel to one another) or around a common axis in an inverted scissor like fashion as shown in FIGS. 3-8 below.

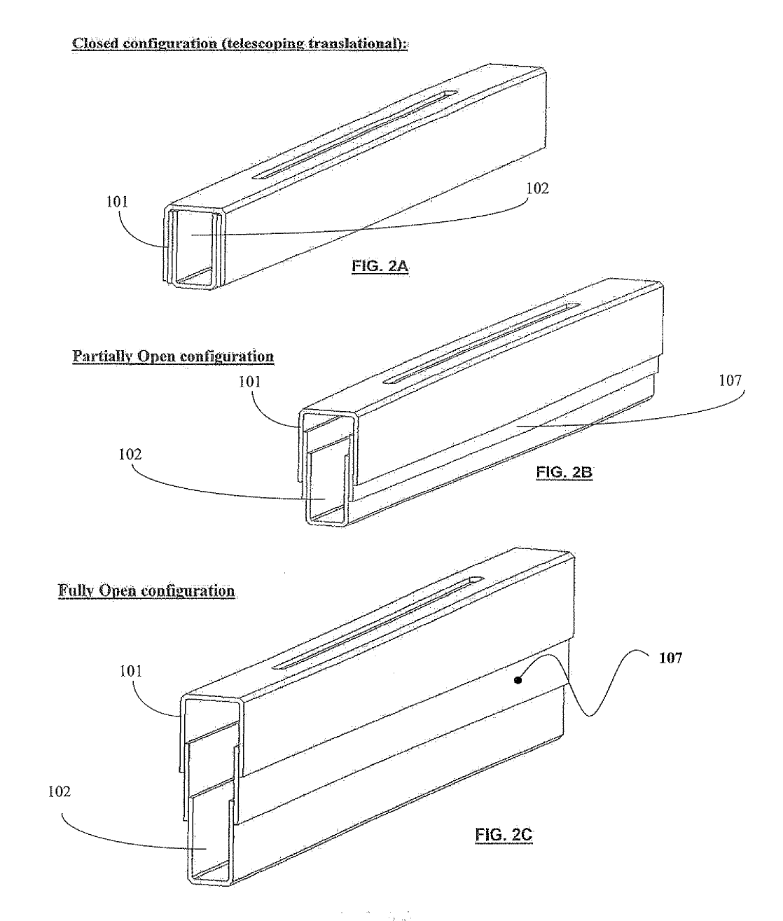

FIG. 2a illustrates an expanding conduit in a closed configuration, FIG. 2b illustrates the expanding conduit in a partially open configuration and FIG. 2c illustrates the expanding conduit in a fully open configuration. Optionally, an additional profile, or profiles, 107 may be placed between the upper 101 and lower 102 profiles in order to enlarge the elongated conduit's working channel in the expanded configuration. On the profiles, there may be visible marks, such as a numerical scale (not shown) to indicate the distance the profiles become separated during expansion. Additionally, in relation to the actuation mechanism, various options are described herein below, may be calibrated to indicate the degree of expansion during actuation. The profiles are connected in a way that their motion is limited to the optimal/desired length of travel. The profiles connection preferably provides a locking arrangement (for example, a ratchet, locking tabs or other locking arrangement, also not shown) that prevents the profiles from folding back after expansion. The locking arrangement is preferably releasable so that, when desired, the lock may be unlocked and the profiles may return to their initial closed configuration.

FIG. 3 illustrates an inverted scissor like expanding conduit in a closed configuration, according to embodiments of the present invention. The inverted scissor like elongated conduit is expanded by applying a force onto one side of the conduit axis thus ‘see-sawing’ the tips on the other side to separate, thus enlarging the working channel.

FIG. 4 illustrates a side view of the inverted scissor like expanding conduit in a closed configuration, according to embodiments of the present invention. The upper and the lower profiles may have support plates 105 to increase the expanding conduit rigidity.

FIG. 5 illustrates the tip design of the inverted scissor like expanding conduit in a closed configuration, according to embodiments of the present invention. The upper profile and the lower profile are closed such that the outer wall of the upper profile form an external wall of the conduit and the second wall of the upper profile form an internal wall inside the lower profile as shown in FIG. 5. At the tip and/or along the top surface of the profile there may be a single or series of protrusions 106 that can be lodged into the surface that comes in contact with the tip. The shape of the protrusion may be triangular, conical, polyhedron, etc. and the matrix may be square, round, linear or other. It is noted that protrusions similar to 106 may be incorporated in other embodiments of the invention.

FIG. 6 illustrates another tip design of the inverted scissor like expanding conduit in a closed configuration, according to embodiments of the present invention. The upper profile and the lower profile are closed such that the walls of the lower profile form the external walls of the conduit and the upper profile walls are closed inside the lower profile as shown in FIG. 6.

According to embodiments of the present invention, the separation of the profiles in any of the profile configurations may be achieved by various expansion actuation mechanisms, and some non limiting examples are described herein below:

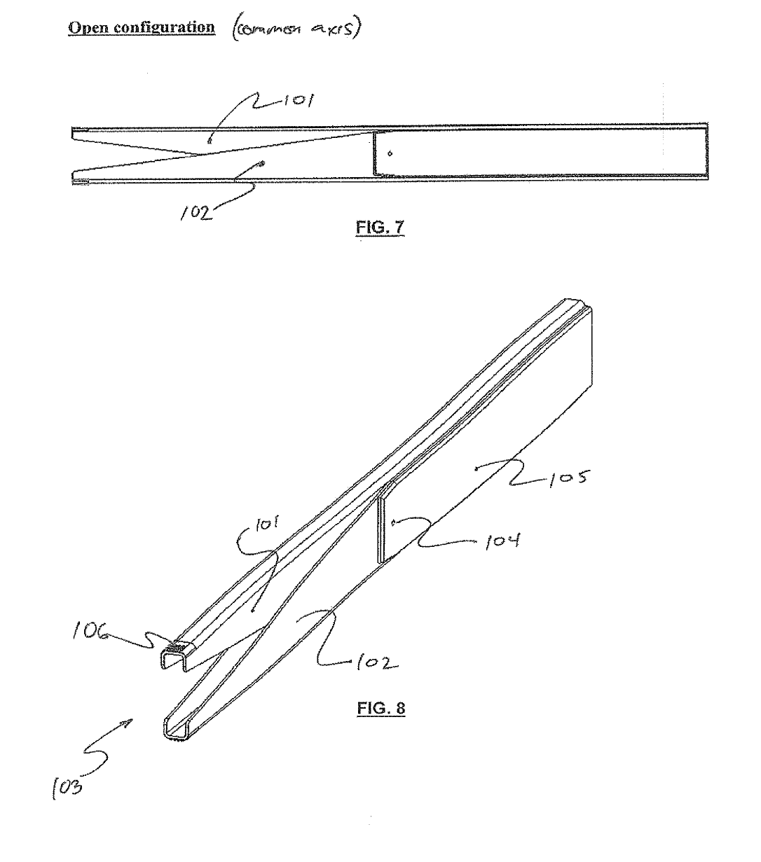

FIG. 7 illustrates an inverted scissor like actuation mechanism of an expanding conduit in an open configuration, according to embodiments of the present invention. After applying a force on to one side of the conduit axis, the tips on the other side, 101 and 102, apply separating forces on two regions of tissue thus enlarging the working channel.

FIG. 8 illustrates a side view of the inverted scissor like actuation mechanism of an expanding conduit in an open configuration, according to embodiments of the present invention. The enlarged working channel 103 is shown with tips 101 and 102 wide open. The inverted scissors like axis 104, support plates 105 and the tip end protrusions 106 are shown in the side view.

FIG. 9 illustrates a cam wedge actuation mechanism of an expanding conduit in a closed configuration, according to embodiments of the present invention. A cam wedge actuation mechanism 200 includes a component 201 with a curved surface 205, an upper rod 202, a lower rod 206 and 2 connecting pins 203 and 204.

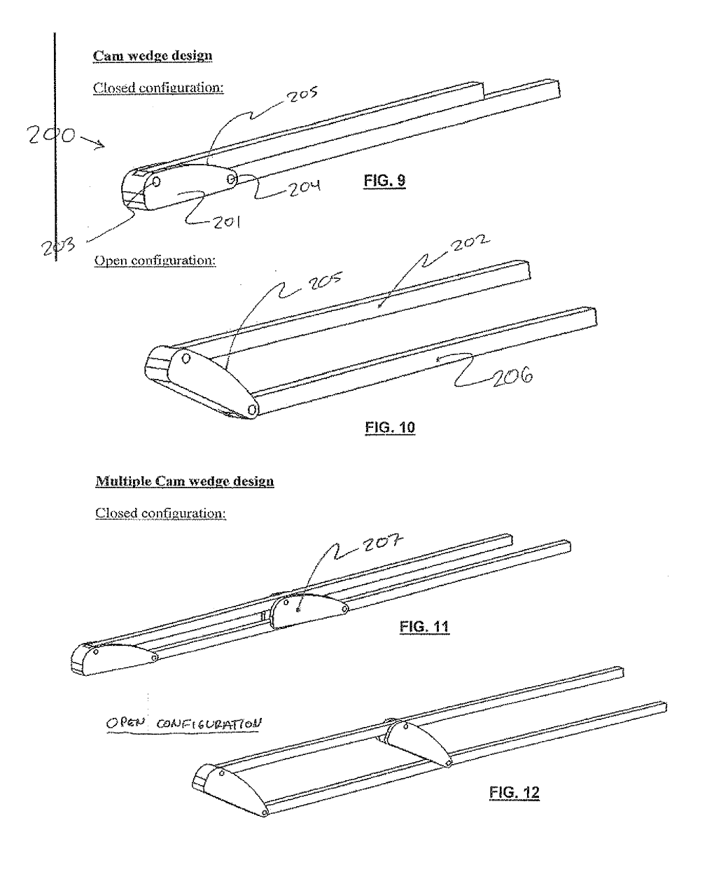

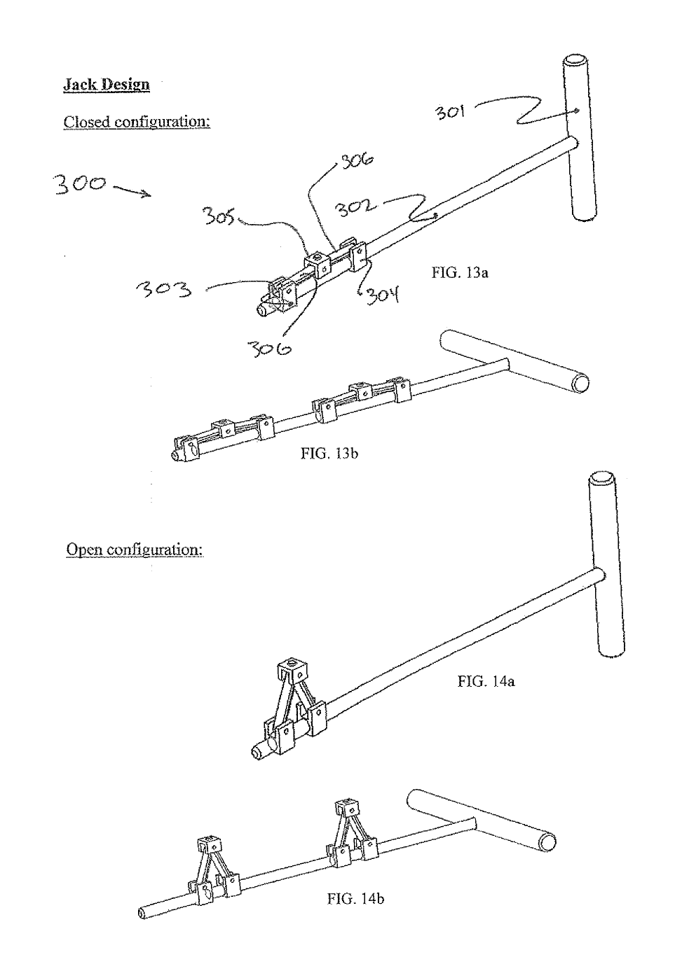

FIG. 10 illustrates the cam wedge actuation mechanism of an expanding conduit in an open configuration, according to embodiments of the present invention. Pulling in or pushing out one of the rods creates a rotation of component 205 around pins 203 and 204. The rotation of component 205 increases the height of the component in the direction perpendicular to the axis of motion and applies force outwards onto the upper and the lower elongated conduit profiles. This perpendicular change in height separates the upper 202 and the lower 206 profiles thereby increasing the height of the working channel (see FIG. 17 and FIG. 18).

FIG. 11 illustrates a multiple cam wedge actuation mechanism of an expanding conduit in a closed configuration, according to embodiments of the present invention. Multiple components 207 may be attached to the rods to facilitate additional points of contact for separating the profiles. Multiple cam wedge components allow applying stronger separating forces (and varied distribution of the force along the profiles) between the two separated regions of tissue over a longer separation distance.

FIG. 12 illustrates the multiple cam wedge actuation mechanism of an expanding conduit in an open configuration, according to embodiments of the present invention. As shown, the multiple cam wedge components rotation increases the height of both components 207 in the direction perpendicular to the axis of motion and thus may be used to increase spacing between two regions of tissue and to enlarge the created working channel.

FIG. 13a illustrates a jack actuation mechanism for an expanding conduit in a closed configuration, according to embodiments of the present invention. A Jack actuation mechanism 300 consisting of a handle 301 attached to a threaded rod 302, a block fixed on the rod 303, a block that moves relative to the rod 304, a block that contacts the upper conduit profile 305 and 2 connector shafts between the blocks 306.

FIG. 13b illustrates a multiple jack actuation mechanism for an expanding conduit in a closed configuration, according to embodiments of the present invention. Again, multiple jack actuation mechanism allows applying stronger separating forces over a longer separation distance between two regions of tissue.

FIG. 14a illustrates the jack actuation mechanism for an expanding conduit in an open configuration, according to embodiments of the present invention. When the handle is turned the movable block 304 advances toward the fixed block 303 causing the central block 305 to travel perpendicular to the blocks 304 and 305 thus separating the upper and the lower profiles and enlarging the created working channel. The threaded rod may have multiple jack subassemblies.

FIG. 14b illustrates the multiple jack actuation mechanism for an expanding conduit in an open configuration, according to embodiments of the present invention. The multiple jack actuation mechanism improves the strength and the stability of the expanding conduit.

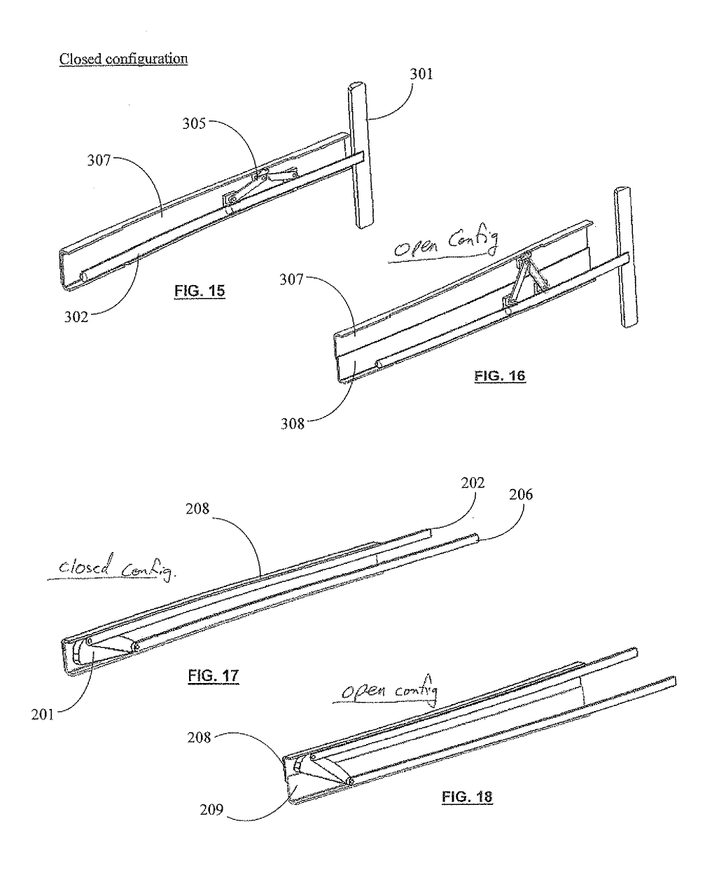

FIG. 15 illustrates a cross section of the jack actuation mechanism inside an expanding conduit in a closed configuration, according to embodiments of the present invention. When actuated, the jack expansion mechanism 305 applies outward forces between the upper 307 and lower 308 profiles so as to expand the elongated conduit as shown in FIG. 16 below. The jack actuation expansion mechanism may be subsequently removed to make the working channel available to access the created space between the two regions of tissue.

FIG. 16 illustrates a cross section of the jack actuation mechanism inside an expanding conduit in an open configuration, according to embodiments of the present invention. The maximal height of the jack actuation mechanism corresponds to the maximal desired height of the expanding conduit in its open configuration and may be varied by the surgeon according to the clinical requirement.

FIG. 17 illustrates a cross section of the cam wedge actuation mechanism inside an expanding conduit 208 in a closed configuration and FIG. 18 illustrates a cross section of the cam wedge mechanism inside an expanding conduit in an open configuration 208 and 209. The maximal height of the cam wedge actuation mechanism corresponds to the length of the earn wedge component 201 and may be varied according to the clinical requirement. The cam wedge actuation mechanism, and the other expansion mechanisms described herein above, may be subsequently removed to make the working channel available to access the created space between the two regions of tissue.

Tissues may be evacuated through the working channel in preparation for an implant insertion to the evacuated space for example. The implant may be a spinal implant inserted into an evacuated disc space between adjacent vertebrae as a non limiting example for the use of the expanding conduit of the present invention.

FIG. 19 illustrates a spring actuation mechanism for an expanding conduit in an closed configuration, according to embodiments of the present invention. An expanding spring design 400 employs a threaded rod 401 connected to a handle 402. On the threaded rod is a curved form 403 made of an elastic material (such as spring steel, Nitinol, polymer, etc). The curved form has one or more arches (symmetric about the rod 401 or just one sided) 404 that come in contact with the upper and lower profiles of the conduit. One end of the curved form is connected to the end of the rod 405 such that the rod has a rotational axis of freedom. At the other end of the curved form there is a threaded block 406.

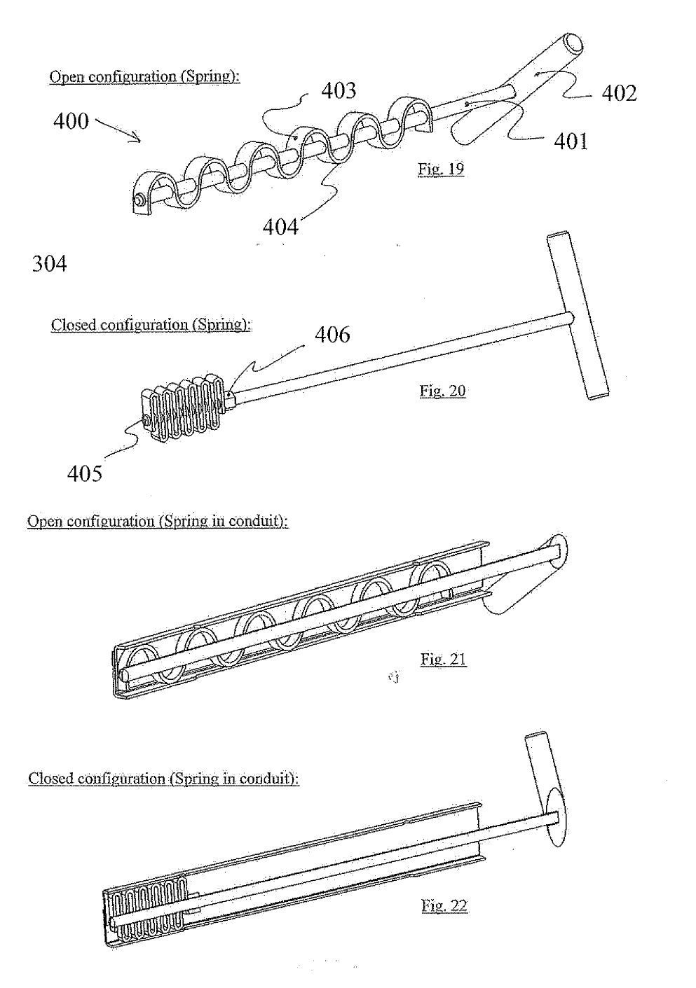

FIG. 20 illustrates the spring actuation mechanism of an expanding conduit in a open configuration, according to embodiments of the present invention. The assembly may be placed in the expanding conduit (any configuration) and when the block 406 is turned, the curved form compresses in such a manner that it pushes outwards the upper and lower conduit profiles. The block may be turned in the opposite direction and the curved form will return to its original position.

FIG. 21 illustrates a cross section of the spring actuation mechanism inside an expanding conduit in a closed configuration, and FIG. 22 illustrates the cross section of the spring actuation mechanism in an open configuration. The expanding spring actuation mechanism 400 may be removed from the conduit. The conduit locking arrangement on the profiles maintains the expanded configuration so that tools and implants may be passed through the enlarged working channel.

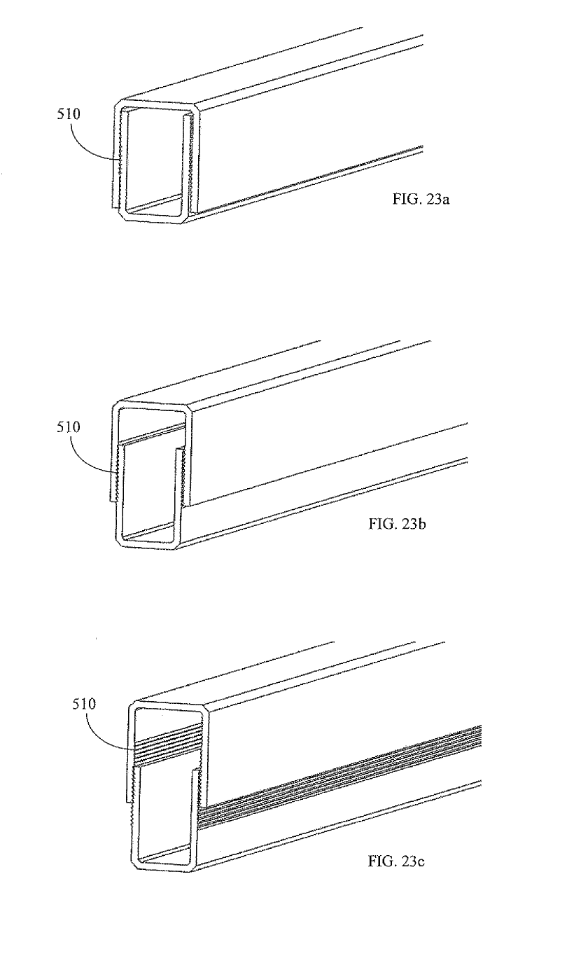

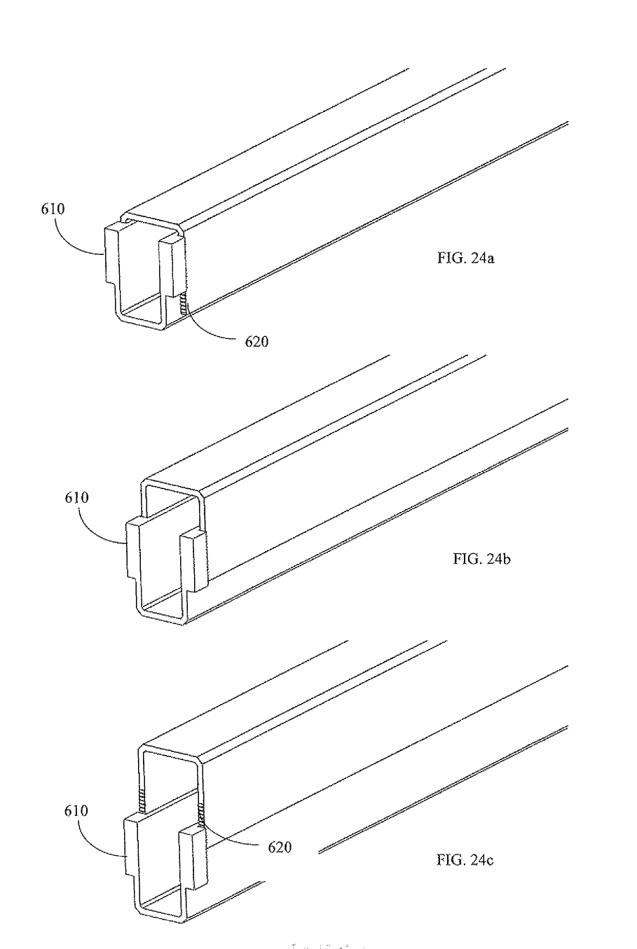

FIGS. 23a-c illustrate a locking arrangement between the upper and the lower profiles of an expanding conduit, according to embodiments of the present invention. The upper and lower profiles have a ratchet like locking arrangement made of matching longitudinal teeth, or protrusions and recesses, 510 such that when the upper and the lower profiles are expanded by one of the optional expansion mechanisms described herein above as shown in FIGS. 23b and 23e, the matching longitudinal protrusions and recesses interlock and prevent contraction back to the conduit closed configuration. The longitudinal ratchet-like locking arrangement thus allows sliding the upper profile upwards smoothly in the expansion direction and allows subsequent removal of an expansion mechanism (such as one of the mechanisms described above) without unintended contraction of the conduit back towards its narrower “closed” state.

FIGS. 24a-c illustrate another locking arrangement between the upper and the lower profiles of an expanding conduit, according to embodiments of the present invention. The ratchet like locking arrangement matching protrusions and recesses are implemented at the expanding conduit upper profile tip front 620 and at the lower profile inner side tip bulge 610. The longitudinal protrusions and recesses are designed to slide smoothly upwards and to prevent sliding back downwards. FIG. 24a shows the expanding conduit tip in its closed configuration, FIG. 24b shows the expanding conduit tip locked in its open configuration and FIG. 24c shows the expanding conduit tip locked in even wider open configuration enlarging the working channel. The ratchet-like function remains effective as long as the upper profile is biased forwards so as to maintain engagement of the ratchet teeth between the profiles. When it is desired to remove the expanding conduit, a release mechanism (not shown) is actuated to allow slight rearward motion of the upper profile, thereby disengaging the ratchet teeth between the profiles and allowing the conduit to return to its collapsed state for easy withdrawal.

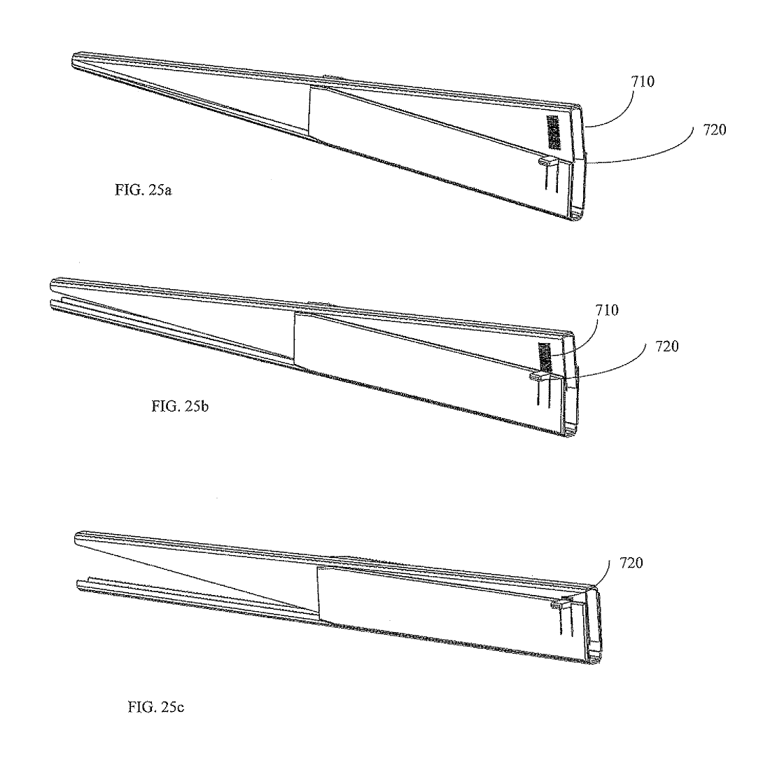

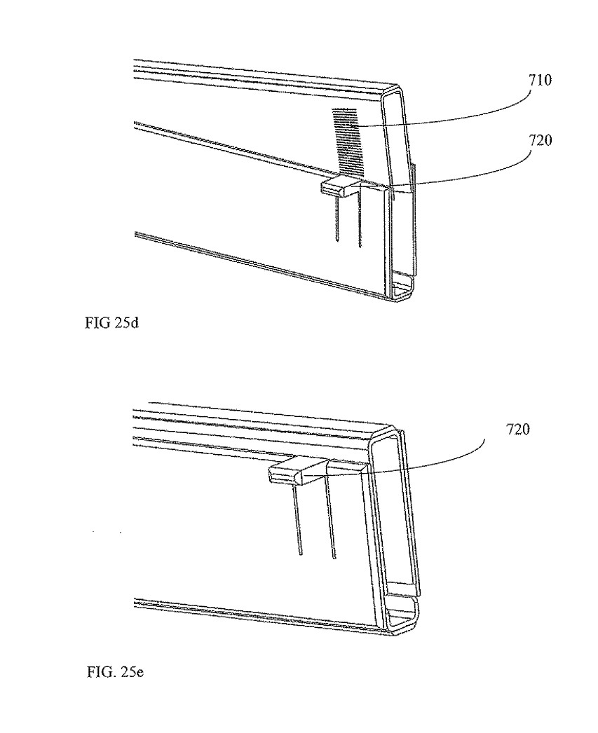

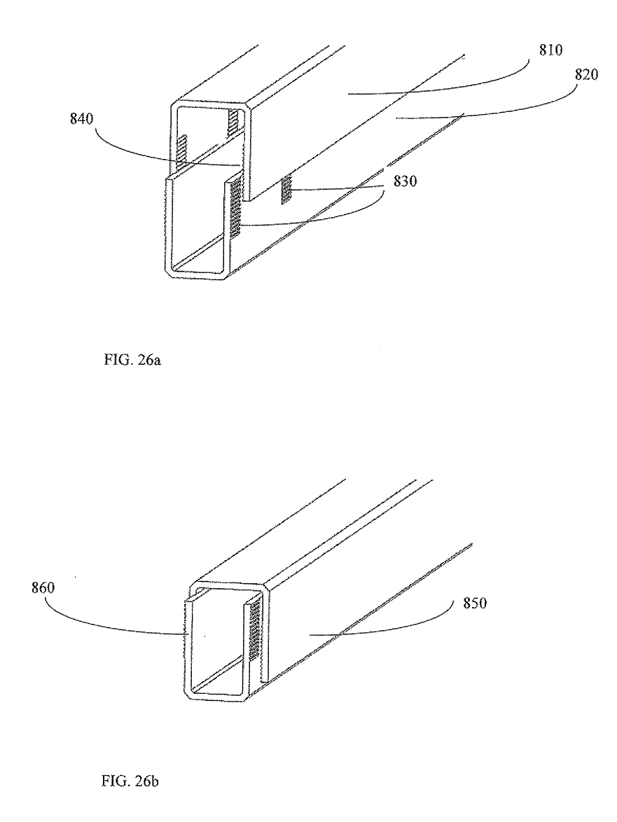

FIGS. 25a-e illustrate a locking arrangement of an inverted scissor like expanding conduit, according to embodiments of the present invention. The upper part of the inverted scissor handle has a socket 710 and the lower part of the inverted scissor handle has a locking button 720 that may be pushed in order to lock into the socket as shown in FIG. 25c. FIG. 25d illustrates in an exploded view the locking arrangement socket 710 and button 720. Socket 710 has asymmetric ridges that allow the button protrusion tooth (not shown) to penetrate and to interlock the asymmetric ridges 710 to button 720 after being rotated around the scissors axis. Ridges 710 are preferably aligned radially relative to the pivot axis between the two profiles. Button 720 may be pulled back to unlock the upper and lower profiles for ease of removal of the conduit.

FIGS. 26a-b illustrate another locking arrangement between the upper and the lower profiles of an expanding conduit, according to embodiments of the present invention, that is a variant of the locking arrangement of FIGS. 23a-23c. The upper 810 and lower 820 profiles have at least one set of matching longitudinal ratchet teeth, or protrusions and recesses, 830 such that when the upper and the lower profiles are expanded by one of the optional expansion mechanisms the matching longitudinal protrusions and recesses interlock and prevent contraction back to the conduit closed configuration. The locking arrangement is configured to be releasable by relative motion of the upper FIG. 26b 850 and lower 860 profiles parallel to a length of the elongated conduit, thereby bringing the locking teeth out of alignment, and thus allowing contraction of the elongated conduit for ease of removal.

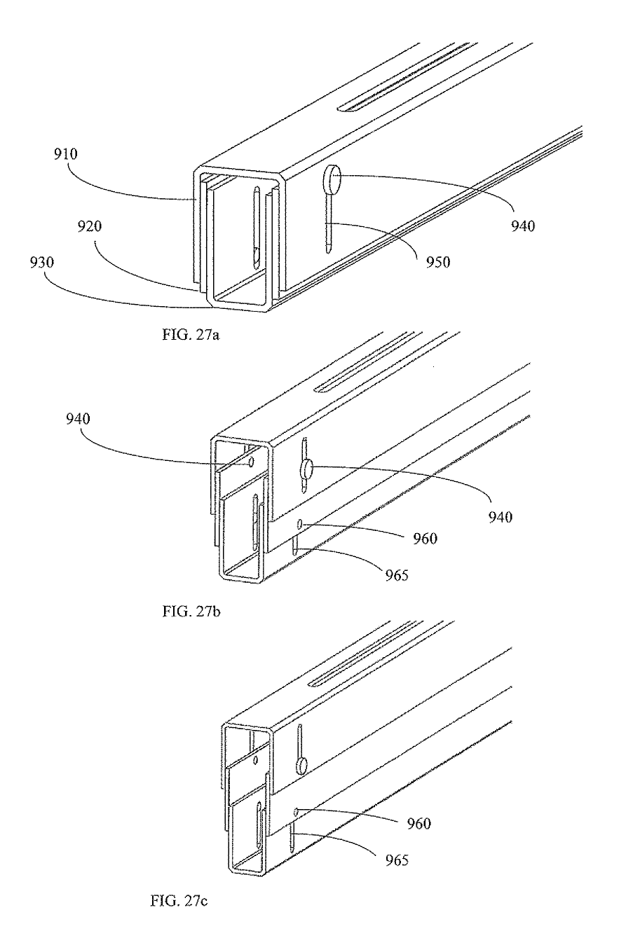

FIGS. 27a-c illustrate an alternative conduit design in which an additional profile is deployed to span a gap between the upper profile and the lower profile, according to embodiments of the present invention. Upper profile 910, additional profile 920 and lower profile 930 are shown in FIG. 27a. Additional profile 930 is typically implemented as two separate planar elements on the two sides of the device. Each planar element supports an outward-projecting pin 940 engaged in a vertical slot 950 of upper profile 910, and an inward-projecting pin 960 engaged in a vertical slot 965 in lower profile 930. This allows the conduit to expand further and to maintain the expanded configuration as shown in FIGS. 27b and 27c. This conduit structure may be implemented with any suitable locking arrangement such as those described herein with reference to FIGS. 23a-23c, 24a-24c or 26a-26b, or with an external expansion mechanism as described below. Depending upon the locking arrangement used, the ratchet teeth or the like may need to be doubled-up in the region of overlap of each of the upper and lower profiles with the additional profile.

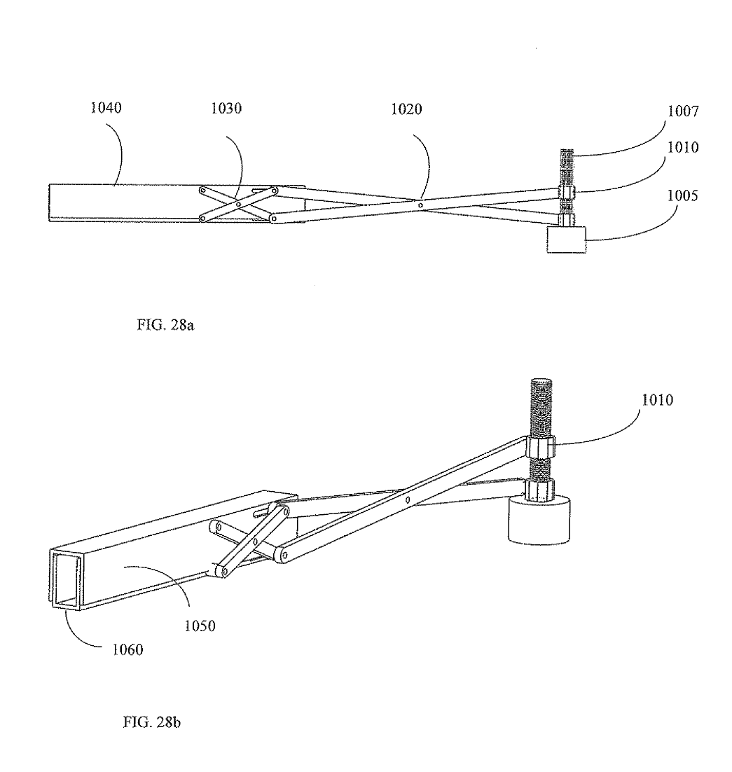

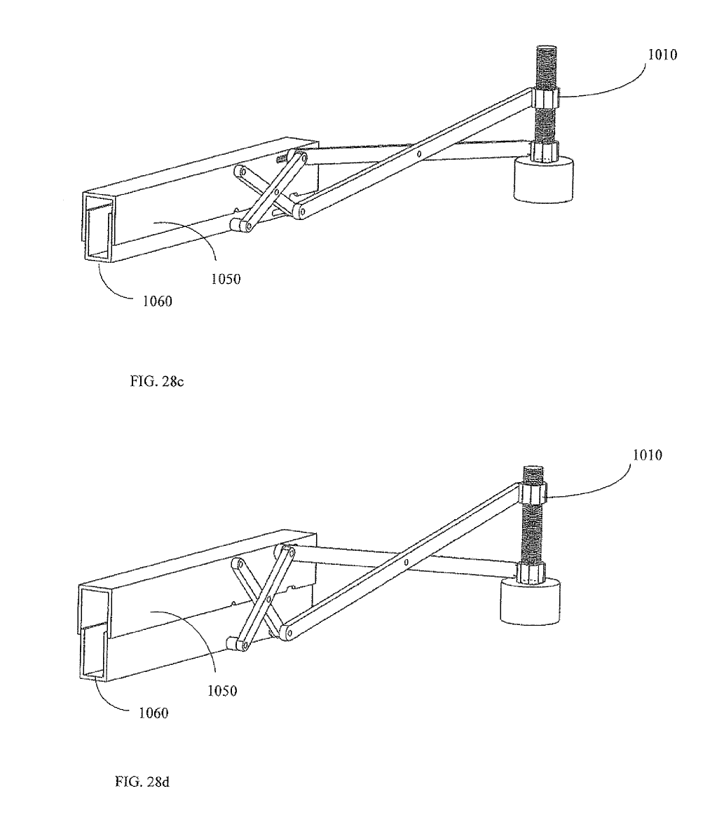

FIGS. 28a-d illustrate an external scissor expansion mechanism and an expanding conduit, according to embodiments of the present invention. FIG. 28a shows an external scissor mechanism 1020 connected to a base 1005 supporting a vertical screw 1007. One leg of external scissor mechanism 1020 can be displaced upwards for example by actuating vertical screw 1007, thereby widening the scissors legs opening angle. Clearly, vertical screw 1007 could alternatively be double-threaded to displace both legs of the scissor mechanism simultaneously in opposite directions, as is known in the art. External scissors 1020 legs are connected to the legs of an expanding conduit scissor mechanism 1030 with pins. Expanding conduit internal scissor mechanism is linked externally to an expanding conduit 1040 at the proximal part that does not enter the body. Provision of second pair of scissor arms 1030 linked to upper profile 1050 and lower profile 1060 ensures that the two profiles remain parallel to each other during expansion. It will be noted that the use of an external expansion mechanism, such as in this non-limiting example, avoids the need for a locking arrangement since the expansion mechanism itself maintains the open state of the conduit without obstructing the working channel through the conduit.

FIG. 28c illustrates the external scissor expansion mechanism and the expanding conduit in the isometric view showing further the upper 1050 and lower 1060 profiles of the expanding conduit in partially expanded configuration. The upper leg 1010 of the external scissors is in partially upwards position. FIG. 28d illustrates the external scissor expansion mechanism and the expanding conduit in the isometric view showing the upper 1050 and lower 1060 profiles of the expanding conduit in a fully expanded configuration. The upper leg 1010 of the external scissors expansion mechanism is in a fully upwards position.

According to the particularly preferred embodiments of the present invention illustrated herein, the expanding conduits have a substantially rectangular cross-section, formed by three-sided upper and lower profiles. However, other upper and lower profiles geometries, such as circular, elliptical or any other profile geometry that may be implemented fall within the general scope of the present invention.

According to certain embodiments of the present invention, the expanding conduits have at least one opening at the tip front and may have alternatively or additionally at least a lateral opening in at least one of the upper or lower profiles upwards, downwards or sideways walls that may be located at the distal portion or a mid portion of the conduits and all combinations and sub combinations of such openings are in the scope of the present invention.

Advantageously, expanding conduits described above may be used to open a space between two regions of tissues and to create a surgical working channel access to the created space between the two regions of tissue through the elongated conduit.

Another advantage of the expanding conduits described above is that an expansion mechanism removably deployable within the conduit is used to increase the spacing between the two regions of tissue.

Another advantage of the expanding conduits described above is that the expansion mechanism may be removed to make the enlarged working channel available to access the space between the separated two regions of tissue.

Another advantage of the expanding conduits described above is that surgical tools, biocompatible material and implants may be introduced through the enlarged working channel.

In summary, expanding conduits described above may be used for opening an internal space between tissues, for distracting and removing tissue, for creating and for expanding a working channel that allows the clinician to perform various clinical procedures through the created working channel, and more specifically for introducing implants and biocompatible material through the created enlarged working channel.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1-20. (canceled)

21. A device comprising:
an elongated conduit comprising:
at least an upper profile; and
at least a lower profile, said upper and lower profiles being relatively displaceable such that, when a distal part of said conduit is deployed between two regions of tissue, a spacing between an upper distal surface of said upper profile and a lower distal surface of said lower profile can be varied, thereby opening a space between the two regions of tissue, and wherein said upper and lower profiles are further configured so as to define therebetween a working channel to provide access to the space between the two regions of tissue through said conduit, said working channel having a vertical dimension at least equal to a vertical spacing between said upper and lower distal surfaces when fully open, and wherein said upper and lower distal surfaces form an extension of upper and lower surfaces of said working channel extending into the tissue.

22. The device of claim 21, further comprising an expansion mechanism removably deployable within said conduit and configured to apply outward forces between said upper and lower profiles so as to expand said elongated conduit, thereby increasing a spacing between the two regions of tissue.

23. The device according to claim 22, wherein said expansion mechanism is deployable within said working channel to apply said outward forces between said upper and lower profiles and is subsequently removed to make said working channel available to access the space between the two regions of tissue.

24. The device according to claim 23, further comprising a locking arrangement configured to lock said upper and lower profiles to maintain an expanded configuration of said elongated conduit after said expansion mechanism is removed.

25. The device according to claim 24, wherein said locking arrangement is configured to be releasable by relative motion of said upper and lower profiles parallel to a length of said elongated conduit, thereby allowing contraction of said elongated conduit for ease of removal.

26. The device according to claim 21, wherein said elongated conduit further comprises one or more additional profiles deployed to span a gap between said upper profile and said lower profile in an expanded configuration of said elongated conduit.

27. The device according to claim 22, wherein said expansion mechanism is an inverted scissor like mechanism having a rotation axis in a mid portion of said elongated conduit, and wherein applying a force onto one side of the conduit separate the tips on the other side of said elongated conduit enlarging said working channel.

28. The device according to claim 22, wherein said expansion mechanism is a cam wedge mechanism comprised of at least one component with a curved surface, an upper and lower rods and connecting pins, and wherein pulling in or pushing out one of the rods creates a rotation of said curved component around said pins and wherein said rotation of said component increases the height of the curved component in the direction perpendicular to the axis of motion and applies force outwards onto said upper and said lower elongated conduit profiles enlarging said working channel.

29. The device according to claim 22, wherein said expansion mechanism is a jack mechanism comprised of at least one set of a handle attached to a threaded rod, a block fixed on the rod, a block that moves relative to the rod, a block that contacts the upper conduit profile and two connector shafts between the blocks, and wherein turning the handle advances said movable block toward said fixed block causing said central block to move in the perpendicular direction to said blocks pushing outwards said upper and lower profiles and enlarging said working channel.

30. The device according to claim 22, wherein said expansion mechanism is a spring mechanism comprised of a threaded rod connected to a handle, an elastic material curved on the threaded rod having one or more arches that come in contact with said upper and lower profiles of said conduit and where one end of said curved elastic material is connected to the end of said rod such that the rod has a rotational axis of freedom and the other end of said curved elastic material is connected to a threaded block, and wherein turning said block compresses said curved elastic material pushing outwards said upper and said lower conduit profiles enlarging said working channel.

31. The device according to claim 21, wherein said upper and lower profiles are hingedly engaged such that pressing together of proximal ends of said upper and lower profiles results in moving apart of distal ends of said upper and lower profiles.

32. The device of claim 21, wherein said conduit has a substantially rectangular cross-section.

33. The device according to claim 21, further comprising a locking arrangement configured to lock said upper and lower profiles in an expanded configuration.

34. A method for accessing a volume within a body, the method comprising the steps of:
(a) inserting into the body an elongated conduit comprising at least an upper profile and a lower profile;
(b) applying outward forces between said upper and lower profiles so as to expand said elongated conduit, thereby increasing a spacing between two regions of tissue and opening a space therebetween; and
(c) accessing said space between the two regions of tissue through said elongated conduit, wherein said working channel has a vertical dimension at least equal to a vertical spacing between said upper and lower distal surfaces when fully open, and wherein said upper and lower distal surfaces form an extension of upper and lower surfaces of said working channel extending into the tissue.

35. The method according to claim 34, wherein said step of applying outward forces is performed using an expansion mechanism removably deployable within said elongated conduit.

36. The method according to claim 25, wherein said expansion mechanism is removed subsequent to said applying outward forces so as to make available a working channel through said elongated conduit to access said space.

37. The method according to claim 36, comprising further the step of removing tissue through said elongated conduit.

38. The method according to claim 37, wherein said removed tissue is tissue of an intervertebral disc.

39. The method according to claim 34, further comprising the step of introducing an implant into said space through said elongated conduit.

40. The method according to claim 34, further comprising the step of introducing biocompatible material into said space through said elongated conduit.
